(19) United States
(12) Patent Application Publication
(10) Pub. No.: US 2024/0366258 A1
(43) Pub. Date: Nov. 7, 2024

(54) KNIFE UNIT

(71) Applicant: Erbe Elektromedizin GmbH, Tuebingen (DE)

(72) Inventor: Felix BOB, Rottenburg (DE)

(21) Appl. No.: 18/650,159

(22) Filed: Apr. 30, 2024

(30) Foreign Application Priority Data
May 4, 2023 (EP) 23171501.2

(51) Int. Cl. A61B 17/3211; A61B 17/28

(52) U.S. Cl. CPC A61B 17/3211; A61B 17/282

(57) ABSTRACT

An instrument with an improved knife unit includes a flat knife arranged in a knife housing. The latter comprises a channel configured for locating the knife therein, wherein on the walls of the channel inwardly projecting projections are formed, preferably in the form of nubs, between which knife is held with play. If knife housing is made of plastic, the projections can be created in a simple manner during primary shaping. If the knife housing is made from a semi-finished product, such as a sheet part by a deformation method, for example by a stamp-bending process, the projections facing one another constricting the channel can be produced in that the walls are provided with cavities on their outer sides. The knife unit is particularly robust against contamination.

### CROSS REFERENCE TO RELATED APPLICATION

This application claims the benefit of European Patent Application No. 23171501.2, filed May 4, 2023, which is incorporated herein by reference in its entirety.

### TECHNICAL FIELD

The invention refers to the knife unit of a surgical instrument as stand-alone unit or as component of this instrument.

### BACKGROUND

Frequently surgical instruments comprise a forceps-like tool part with which tissue can be gripped. Frequently a movably supported knife is assigned to this tool, which serves to cut tissue gripped by the forceps.

For example, US 2016/0051316 A1 discloses for this purpose an instrument configured for the laparoscopic use having a longitudinal shaft at the distal end of which a tool of the indicated configuration is arranged.

The knife, which serves to cut through tissue held in the tool, is guided in the jaws of the forceps-like tool in a laterally curved channel in order to cut tissue, if it is distally forwarded in longitudinal direction.

The two jaws comprise electrodes to which current can be applied in order to heat and thus to coagulate tissue held by the instrument. Thereby the risk exists that tissue sticks on the knife depending on the knife temperature. In order to avoid too excessive heating of the knife by heat transfer from the electrodes onto the knife, teeth are arranged in the flanks of the knife channel, which contact the knife only at small contact surfaces that are distanced from one another in longitudinal direction.

In addition, EP 2 893 886 B1 discloses a blade unit for a single-use scalpel with associated reusable scalpel handle. A blade holder and blade protection are assigned to the blade of the scalpel. In order for the blade holder and handle to slide easily in the blade protection, the latter is provided with a slide rib on the inside extending in longitudinal direction.

Furthermore, US 2022/0331498 A1 discloses an anti-stick coating for cutting elements of electrosurgical cutting tools, such as knives movable in longitudinal direction, which shall cut tissue held between jaws. Such an anti-stick coating can be provided on insulation elements of electrosurgical cutting devices, for example. The hydrophobic structures can limit the contact to minimum areas between the tissue and the insulating elements by means of microscopic projections and depressions in the surface.

Additionally, a knife cartridge for a sealing instrument is known from EP 3 815 642 A1 in which a knife supported movably in longitudinal direction is supported inside an individual cartridge that can be separated from the instrument.

During use of an instrument on the patient the knife can be moved multiple times in longitudinal direction whereby tissue liquid can be displaced into the knife channel. If the knife is then not operated for a certain time, it can tend to stick so that it can only be moved with difficulty or not at all. This can interfere with the operation process sustainably.

### SUMMARY

It is one the object of the invention to provide an improved instrument, particularly an improved knife unit.

This object is solved by means of the knife unit as described herein.

The knife unit according to the invention comprises a knife housing having a channel extending in longitudinal direction for locating the knife therein. The channel has a length sufficient to completely locate the strip-shaped knife therein, so that it does not or only slightly project out of an end of the channel. On the other hand, the knife has a sufficient length so that it can be moved sufficiently far out of the channel in longitudinal direction for cutting through biological tissue, so that it passes through the entire knife slot, whereby a rear (proximal) part of the knife still remains inside the channel, for example in the knife slot of jaws of a forceps-like instrument.

The knife housing can be part of the surgical instrument, a knife cartridge or a knife cassette, which is releasably connected with the instrument. The cartridge or cassette can be configured according to the model of EP 3 815 642 A1 or also in another manner. If the knife housing is part of the instrument, the channel transitions into the above-mentioned knife slot preferably smooth and without steps. If the knife housing is, however, part of a knife cassette or knife cartridge, attachment means are provided on the instrument, which hold the knife cassette or knife cartridge so that the channel of the knife housing smoothly adjoins the knife slot of the instrument.

The knife housing comprises projections inside its channel on those surfaces facing the flanks of the knife. These projections have the effect that a minimum distance between the surfaces of the channel and the flanks of the knife is always maintained. If the knife is moved forward and backward in longitudinal direction during cutting of tissue and if secretion, tissue residues or the like are displaced into the channel, it is guaranteed, due to the minimum distance between the surfaces and the flanks guaranteed by means of the projections, that the knife does not stick inside the channel. Also, if the instrument is at first put aside during surgery and is again used after a shorter or longer period, no sticking of the knife and thus an impairment of the instrument use can occur.

Preferably the knife is entirely located in the knife housing in the rest position. In doing so, the edge of the knife is protected. In addition, the knife unit can reduce or eliminate the risk of injury for operating personnel, if it is configured as stand-alone knife cassette or knife cartridge.

Preferably the knife is arranged inside the channel with play, particularly with lateral play. The play has an amount of preferably multiple hundredths of a millimeter, wherein the height of the projections extending from the other surface of the channel has an amount of preferably at least one tenth of a millimeter. In this manner capillary effects are largely inhibited, which otherwise could result in easy entrance of body liquids, secretion, particularly albuminous liquids, into the channel.

Preferably the knife is made of metal, for example stainless steel. Likewise, the knife housing can preferably be made of metal or alternatively of plastic. The arrangement of the projections at the lateral surfaces of the channel allows the use of simple and reliable manufacturing technologies. For example, the knife can be produced as flat part without plastic deformation, for example by means of appropriate separation methods out of flat material. On the contrary, the knife housing can be created, for example, as stamp-bent part, whereby the projections extending inwardly can be produced by embossing, for example.

However, the knife housing can also be configured from plastic. Independent therefrom only a local contact is established between the projections and the flanks of the knife, for example a point contact or a contact along lines, particularly in longitudinal direction (movement direction) extending lines, preferably particularly interrupted lines. The projections are thereby preferably rounded and thus transition without step into the respective surfaces. This reduces potential friction between the knife and the knife guide, particularly if tissue residues or secretion are present inside the channel.

### BRIEF DESCRIPTION OF THE DRAWINGS

Further details of advantageous embodiments of the invention are derived from the dependent claims, the description and the associated drawing. The drawing shows:

FIG. 1 an instrument having a knife unit according to the invention in schematic perspective illustration,

FIG. 2 an instrument with removable knife unit according to the invention in perspective schematic illustration,

FIG. 3 the knife unit of an instrument according to FIG. 1 or 2 in schematic perspective illustration,

FIG. 4 the knife unit according to FIG. 3 in longitudinal section explosion illustration,

FIG. 5 the knife unit according to FIG. 3 in cross-section illustration,

FIG. 6 the knife unit according to FIG. 5 in longitudinal section illustration in part.

### DETAILED DESCRIPTION

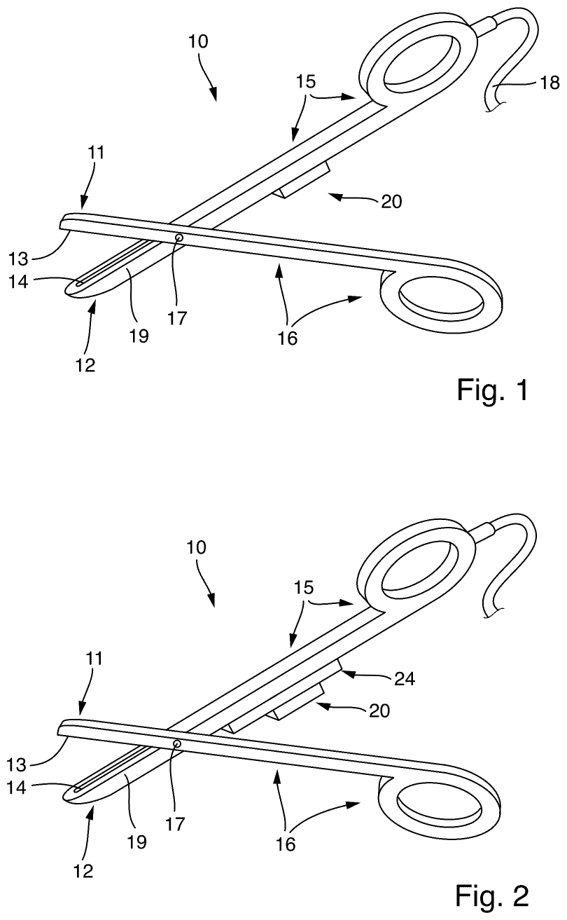

An instrument 10 is illustrated in FIG. 1, which is configured to hold, coagulate and separate tissue by means of two jaws 11 and 12. For this purpose, the jaws 11, 12 are provided with electrodes 13, 14 on their sides facing one another in order to heat and thus coagulate tissue held therebetween by means of passing electrical current therethrough. The opening and closing of jaws 11, 12 is carried by means of handles 15, 16 into which the jaws 11, 12 transition and are connected to one another in a hinged manner at a hinge 17. The electrical power required for applying current to the electrodes 13, 14 and thus the tissue can be supplied via a cable 18 from a generator.

In at least one of the jaws 11, 12, preferably in both, a knife slot 19 is arranged respectively. By means of a suitable operating element, for example a slider 20 arranged on one of the handles 15, 16 a knife 21 can be moved in the knife slot 19. A suitable operating device serves for operation of the latter, such as the slider 20, which is arranged on one of the handles 15, 16.

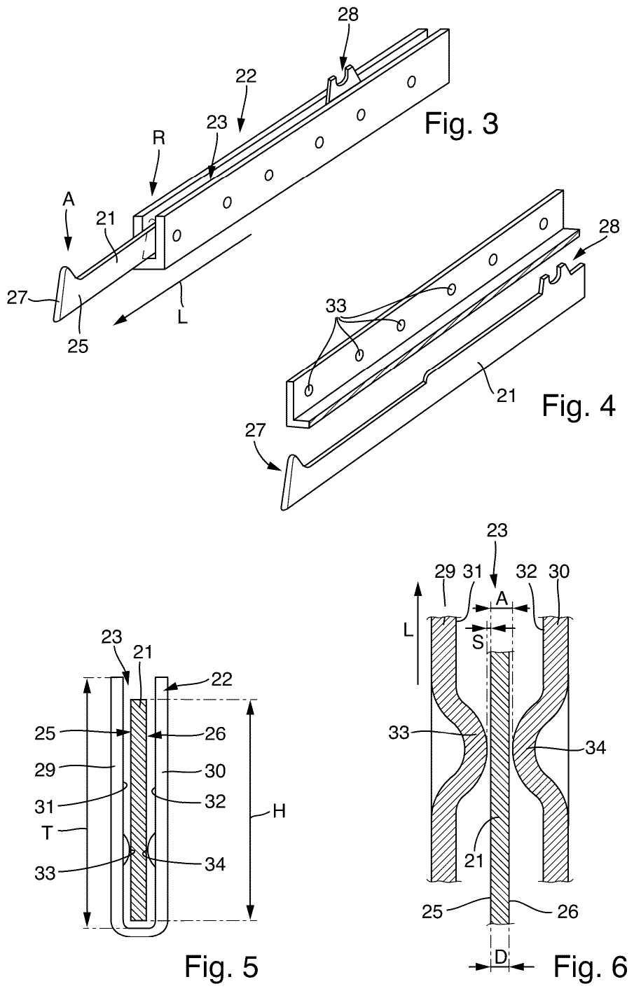

The knife 21 is illustrated in FIGS. 3, 4, 5 and 6 in different contexts individually. The knife 21 is arranged in a knife housing 22 in a channel 23 defining a longitudinal direction L.

The knife housing 22 can be part of the handle 15 and can be thus non-releasably connected with the instrument 10. Alternatively, the knife housing 22 can be part of a knife cassette 24, which is removably arranged on one of the handles 15, 16 in order to be able to exchange the knife cassette 24 and together with the latter the knife 21 after use. Such an instrument 10′ is schematically depicted in FIG. 2. It can be particularly a reusable sterilizable instrument 10′, which is connected prior to the use with a not re-sterilizable and therefore non-reusable, however, sterilely provided knife cassette 24.

For operating knife 21, i.e. for moving the knife 21 in longitudinal direction L, again a slider 20 can be provided directly on the cassette or any other suitable operating element can be provided at another position of instrument 10′. Apart therefrom the description provided in the context of FIG. 1 applies accordingly for instrument 10′ of FIG. 2 on the basis of the already introduced reference signs.

In the instrument 10 according to FIG. 1 as well as in the instrument 10′ according to FIG. 2, knife 21 and knife housing 22 are configured according to the principles shown in FIGS. 3 to 6.

The knife 21 is preferably a flat strip-shaped sheet part of uniform thickness having two flat sides forming flanks 25, 26 facing away from one another. On a face end of knife 21 a cutting edge 27 is formed, which can be orientated transverse and thus obliquely or orthogonal to the longitudinal direction L. Apart therefrom the knife 21 is preferably planar on both flanks 25, 26 and entirely or at least largely configured without interruptions, i.e. without openings, slits or the like.

On its end opposite the cutting edge 27, knife 21 can comprise a coupling structure 28 in order to be specifically moved in longitudinal direction L or against longitudinal direction L by means of an operating mechanism, for example in form of slider 20. The coupling structure 28 can be a mouth-like cavity opening transverse to the longitudinal direction L, a projection or the like.

For example, the knife housing 22 can be a bent sheet part having a U-shaped cross-section, for example a stamp bent part, or also a plastic part. It comprises two walls 29, 30 with surfaces 31, 32 facing one another. On the surfaces 31, 32 projections 33, 34 are arranged facing one another and holding the knife 21 with play therebetween. The projections 33, 34 can be formed by means of depressions formed from the outside into the respective wall 29, 30, as illustrated by FIG. 6. The projections 33, 34 form inwardly orientated nubs, for example in the form of rounded elevations. Between two oppositely arranged nubs 33, 34 a distance A is defined. The latter is longer than a thickness D of knife 21, which is to be measured between its flanks 25, 26. The difference between a distance A and thickness D is the play S, which has an amount of preferably approximately 0.05 mm. It can also be larger, for example 0.1 mm or smaller, for example only 0.03 mm. The projections 33, 34 preferably elevate approximately about an absolute value of 0.1 mm beyond the respective surface 31, 32. The distance A is preferably approximately 0.2 mm with a thickness D of knife 21 of approximately 0.15 mm, for example.

The knife unit consisting at least of knife 21 and knife housing 22 described so far operates in connection with instrument 10 or 10′ according to FIG. 1 or 2 as follows:

For coagulation and cutting of tissue, for example blood vessels or other vessels or other biological tissue, it is held by closing the jaws 11, 12 and applied with current by means of electrodes 13, 14 in order to create a coagulation effect. If the latter is sufficiently well advanced, the tissue can be cut by means of the cutting edge 27 of knife 21. The knife 21 is transitioned for this purpose by operating slider 20 or another suitable operating organ, at least partly out of the rest position R illustrated in dashed lines in FIG. 3 into the active position A. Thereby the distal part of knife 21 leaves channel 23 and runs in the knife slot 19 between the closed jaws 11, 12 in direction toward the distal end thereof. Thereby knife 21 cuts through the tissue. After completion of the cut, the knife 21 can be moved again out of knife slot 19 and into the channel 23. If instrument 10 or 10′ is opened again, the ends, for example ends of a blood vessel, which are now separated from one another and sealed due to the coagulation process, are released.

During an operation this process can be repeated multiple or many times. Thereby knife 21, which has contacted biological tissue with its flanks 25, 26, can displace secretion and tissue into channel 23. In addition, due to the operation in humid environment, liquid can reach the knife housing 22. The projections 33, 34, however, avoid formation of a too narrow gap between the surfaces 31, 32 and the flanks 25, 26 of knife 21 arranged opposite thereto. The formation of capillary gaps is avoided, which would otherwise support the entrance of liquids into channel 23. In addition, the projections 33, 34 avoid that knife 21 becomes difficult to move, gets caught or even sticks, even if liquid has entered channel 23.

Due to their smooth curvature, the projections 33, 34 are suitable to serve as stripper for solidified or toughened tissue liquid present on the knife. Thereby it is particularly useful that the projections 33 are arranged on a common line in longitudinal direction L, i.e. in movement direction of knife 21 successively, as apparent from FIG. 4, whereby their dimensions orthogonal to the movement direction L (with view in vertical direction in FIG. 5) are remarkably smaller than the depth T of channel 23 or height H of knife 21 measured in the same direction.

The projections 33 distanced in longitudinal direction L, particularly in form of nubs, are able to wipe away material sticking on the flanks 25, 26 of knife 21 and to collect it in the interstice between flank 25 and the surface 31 harmlessly. The same applies for the projections 34 and the flank 26 as well as the surface 32. In the result, knife 21 remains smoothly movably in longitudinal direction and indeed also, if tissue liquid has entered channel 23 and has possibly fixed on knife 21. In addition, the tendency of entrance of liquids into channel 23 is reduced.

An instrument 10 with improved knife unit comprises a flat knife 21 arranged in a knife housing 22. The latter comprises a channel 23 configured for locating the knife 21 therein, wherein on the walls of the channel 23 inwardly projecting projections 33, 34 are formed, preferably in the form of nubs, between which knife 21 is held with play. If knife housing 22 is made of plastic, the projections 33, 34 can be created in a simple manner during primary shaping. If the knife housing 22 is made from a semi-finished product, for example a sheet part by a deformation method, for example by a stamp-bending process, the projections 33, 34 facing one another constricting the channel 23, can be produced in that the walls 29, 30 are provided with cavities on their outer sides. The knife unit according to the invention is particularly robust against contamination.

### LIST OF REFERENCE SIGNS

10, 10′ instrument
11, 12 jaws
13, 14 electrodes
15, 16 handles
17 hinge
18 cable
19 knife slot
20 slider
21 knife
22 knife housing
23 channel
L longitudinal direction
24 knife cassette
25, 26 flanks of knife 21
27 cutting edge
28 coupling structure
29, 30 walls of knife housing 22
31, 32 surfaces (interior surfaces) of walls 29, 30
A distance
D thickness of knife 21
S lateral play of the knife inside channel 23
33, 34 projections
T depth of channel 23
H height of knife 21

1. A knife unit on or for a medical or surgical instrument (10, 10′), comprising:
a knife (21) comprising a strip-shaped body having two flat sides (25, 26) facing away from one another;
a knife housing (22) comprising a channel (23) in which the knife (21) is movably arranged in a longitudinal direction (L) between a rest position (R) and an active position (A);
wherein the knife (21) is partly moved out of the knife housing (22) in the active position (A), and
wherein the knife housing (22) comprises projections (33, 34) in the channel (23) on surfaces (31, 32) of the channel facing the sides (25, 26) of the knife (21).

2. The knife unit according to claim 1, wherein the knife (21) is entirely located inside the knife housing (22) in the rest position (R).

3. The knife unit according to claim 1, wherein the knife (21) is arranged with play (S) in the channel (23).

4. The knife unit according to claim 1, wherein the surfaces of the channel (23) comprise two planar surfaces that face one another.

5. The knife unit according to claim 1, wherein the knife (21) is made of metal.

6. The knife unit according to claim 1, wherein a blade (27) is formed on an end of the knife (21).

7. The knife unit according to claim 6, wherein the blade (27) is arranged transverse to the longitudinal direction (L).

8. The knife unit according to claim 1, wherein a coupling structure is disposed on the knife (21) which is configured for connection to a driving device (20).

9. The knife unit according to claim 1, wherein the projections (33) comprise a plurality of projections on one of the surfaces of the channel, wherein the plurality of projections are aligned along a common straight line oriented parallel to the longitudinal direction (L).

10. The knife unit according to claim 1, wherein the projections (33, 34) are rounded elevations on the surfaces (31, 32).

11. The knife unit according to claim 1, wherein line contacts or point contacts are established between the projections (33, 34) and the sides (25, 26) of the knife.

12. The knife unit according to claim 1, wherein the projections (33, 34) are nubs.

13. The knife unit according to claim 12, wherein a distance (A) between two nubs arranged opposite from one another is larger than a thickness (D) of the knife measured between the sides (25, 26) of the knife (21), wherein a difference between the distance (A) and the thickness (D) of the knife corresponds with the play in the channel.

14. The knife unit according to claim 1, wherein the projections (33, 34) comprise a plurality of projections on each of the surfaces of the channel, wherein the plurality of projections on each surface of the channel are aligned along a line oriented parallel to the longitudinal direction (L).

15. The knife unit according to claim 1, wherein the knife housing (22) is part of an exchangeable cartridge (24).
